(19) United States
(12) Patent Application Publication
(10) Pub. No.: US 2019/0221291 A1
(43) Pub. Date: Jul. 18, 2019

(54) Apparatuses And Systems Utilizing Zeta Potential Prediction of Structures and Methods of Use Thereof

(71) Applicant: Rutgers, The State University of New Jersey, New Brunswick, NJ (US)

(72) Inventors: Daniel Grisham, Piscataway, NJ (US); Vikas Nanda, Highland Park, NJ (US)

(73) Assignee: Rutgers, The State University of New Jersey, New Brunswick, NJ (US)

(21) Appl. No.: 16/249,348

(22) Filed: Jan. 16, 2019

Related U.S. Application Data
(60) Provisional application No. 62/617,702, filed on Jan. 16, 2018.

(51) Int. Cl. G16C 20/20; G16C 20/30

(52) U.S. Cl. CPC G16C 20/20; G16C 20/30

(57) ABSTRACT

Various embodiments of the present disclosure provide for an exemplary method that includes: obtaining a modified compound having an improved dissolution in a solution than an original compound; where the modified structure is determined based at least in part on: i) sampling a plurality of molecular conformations of at least one of: 1) the original compound or 2) a plurality of candidate structures; where each candidate structure differs from the original compound in a conformational change, a structural change, or both; comparing, by a processor, average zeta potentials of the plurality of candidate structures among each other and to an average zeta potential of the original compound; and determining, by the processor, based on the comparing, a desired candidate structure, expected to have a higher solubility in the solution; and adapting a desired compound having the at least one desired candidate structure to the solution.

### RELATED APPLICATIONS

This application claims priority of U.S. Provisional Application No. 62/617,702, filed Jan. 16, 2018, the entirety of which is incorporated herein by reference for all purposes.

### FIELD OF INVENTION

The present disclosure generally relates to apparatuses and systems utilizing zeta potential prediction of structures and methods of use thereof.

### BACKGROUND

Prediction of zeta potential is useful in identifying stable formulations of drugs. Currently there are methods for calculating zeta potential from measured electrophoretic mobility using the Helmholtz-Smoluchowski equation for electrophoresis. Some exemplary methods are Zeta (free on sourceforge), Mobility/Winmobil (University of Melborne), Software with zetameters (Malvern, Brookhaven etc.), and ZEECOM (Microtec Co., Ltd) However, modeling electrophoretic mobility of molecular structures is only applicable for calculating the zeta potential during electrophoresis and such methods lack the ability to estimate the position from the molecular surface, where the zeta potential is defined.

### SUMMARY

Various embodiments of the present disclosure provide for an exemplary method that at least include the following steps: obtaining at least one modified compound having a modified structure; where the at least one modified compound is related to an original compound having an original structure; where the modified structure differs from the original structure; where the at least one modified compound having the modified structure has an improved dissolution in a solution than the original compound having the original structure; where the modified structure is determined based at least in part on: i) sampling a plurality of molecular conformations of at least one of: 1) the original compound having the original structure or 2) a plurality of candidate structures; where each candidate structure differs from the original compound in at least one conformational change, at least one structural change, or both; ii) for each molecular conformation of a respective sampled structure: 1) estimating, by a processor, a hydrodynamic radius; 2) estimating, by the processor, a slip plane position by subtracting a radius of the sampled structure from the estimated hydrodynamic radius; 3) assigning, by the processor, atomic charges and radii to the respective molecular conformation of the respective sampled structure; 4) determining, by the processor, potentials of the respective molecular conformation of the respective sampled structure in a simulated solution environment of the solution, at a solvent-excluded surface (SES) on the respective sampled structure inflated to the estimated slip plane which coincides with the estimated hydrodynamic radius or a measured hydrodynamic radius; and 5) calculating a zeta potential of the respective molecular conformation of the respective sampled structure by averaging the determined potentials at the inflated SES; iii) calculating, by the processor, an average zeta potential of each sampled structure by averaging the calculated zeta potentials of the plurality of molecular conformations of the respective sampled structure; iv) comparing, by the processor, average zeta potentials of the plurality of candidate structures among each other and to an average zeta potential of the original compound; and v) determining, by the processor, based on the comparing at step (iv), at least one desired candidate structure; vi) where the at least one desired candidate structure, based on a respective average zeta potential, is expected to have a higher solubility in the solution than at least one other candidate structure of the plurality of candidate structures and the original compound; vii) where the at least one desired candidate structure is the modified structure of the modified compound; and viii) adapting a desired compound having the at least one desired candidate structure to the solution.

In various embodiments of the present disclosure, the exemplary method further includes: determining, by the processor, one or more solution conditions of the solution.

In various embodiments of the present disclosure, the original compound is a protein.

In various embodiments of the present disclosure, the original compound is an antibody.

In various embodiments of the present disclosure, the original compound is a catalyst.

In various embodiments of the present disclosure, the original compound is an enzyme.

In various embodiments of the present disclosure, the sampling the plurality of the molecular conformations of the sampled structure further includes: preparing the sampled structure for a molecular dynamics simulation; optimizing, by the processor, a geometry of the sampled structure by energetically minimizing the sampled structure via a first molecular dynamics simulation; thermally exciting the sampled structure; performing, by the processor, a second molecular dynamics simulation with the sampled structure in the solution until the sampled structure reaches a steady-state; and sampling, by the processor, the plurality of respective molecular conformations of the sampled structure.

In various embodiments of the present disclosure, the determining the at least one desired candidate structure further includes: selecting, by the processor, the at least one desired candidate structure from the plurality of candidate structures.

In various embodiments of the present disclosure, the determining the at least one desired candidate structure further includes: identifying, by the processor, the at least one conformational change, the at least one structural change, or both, to be made to at least one particular candidate structure of the plurality of candidate structures.

In various embodiments of the present disclosure, the slip plane position is determined based, at least in part, on the estimated hydrodynamic radius.

Various embodiments of the present disclosure provide for an exemplary system that at least include the following components: at least one specialized computer machine, including: a non-transient memory, electronically storing particular computer executable program code; and at least one computer processor which, when executing the particular program code, becomes a specifically programmed computer processor configured to at least: determine a modified structure of at least one modified compound; where the at least one modified compound is related to an original compound having an original structure; where the modified structure differs from the original structure; where the at least one modified compound having the modified structure has an improved dissolution in a solution than the original compound having the original structure; where the determination of the modified structure of at least one modified compound includes: i) receiving sampling data of sampling a plurality of molecular conformations of at least one of: 1) the original compound having the original structure or 2) a plurality of candidate structures; where each candidate structure differs from the original compound in at least one conformational change, at least one structural change, or both; ii) for each molecular conformation of a respective sampled structure and based on the sampling data: 1) estimating a hydrodynamic radius; 2) estimating a slip plane position by subtracting a radius of the sampled structure from the estimated hydrodynamic radius; 3) assigning atomic charges and radii to the respective molecular conformation of the respective sampled structure; 4) determining potentials of the respective molecular conformation of the respective sampled structure in a simulated solution environment of the solution, at a solvent-excluded surface (SES) on the respective sampled structure inflated to the estimated slip plane which coincides with the estimated hydrodynamic radius or a measured hydrodynamic radius; and 5) calculating a zeta potential of the respective molecular conformation of the respective sampled structure by averaging the determined potentials at the inflated SES; iii) calculating an average zeta potential of each sampled structure by averaging the calculated zeta potentials of the plurality of molecular conformations of the respective sampled structure; iv) comparing average zeta potentials of the plurality of candidate structures among each other and to an average zeta potential of the original compound; and v) determining based on the comparing at step (iv), at least one desired candidate structure; vi) where the at least one desired candidate structure, based on a respective average zeta potential, is expected to have a higher solubility in the solution than at least one other candidate structure of the plurality of candidate structures and the original compound; vii) where the at least one desired candidate structure is the modified structure of the modified compound; and viii) at least one apparatus configured to adapt a desired compound having the at least one desired candidate structure to the solution.

### BRIEF DESCRIPTION OF THE DRAWINGS

Various embodiments of the present disclosure, briefly summarized above and discussed in greater detail below, can be understood by reference to the exemplary embodiments of the present disclosure depicted in the appended drawings. It is to be noted, however, that the appended drawings illustrate only exemplary embodiments of the present disclosure and are therefore not to be considered limiting of its scope and other equally effective embodiments are possible.

FIGS. 1-12 illustrate certain aspects in accordance with some embodiments of the present disclosure.

To facilitate understanding, identical reference numerals have been used, where possible, to designate identical elements that are common to the exemplary figures. The exemplary figures are not drawn to scale and may be simplified for clarity. It is contemplated that elements and features of one embodiment may be beneficially incorporated in other embodiments without further recitation.

### DETAILED DESCRIPTION

Among those benefits and improvements that have been disclosed, other objects and advantages of various embodiments of the present disclosure can become apparent from the following description taken in conjunction with the accompanying figures. Detailed embodiments of the present disclosure are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though they may. Furthermore, the phrases “in another embodiment” and “in some other embodiments” as used herein do not necessarily refer to a different embodiment, although they may. Thus, as described below, various embodiments of the present disclosure may be readily combined, without departing from the scope or spirit of the present disclosure. Further, when a particular feature, structure, or characteristic is described in connection with an implementation, it is submitted that it is within the knowledge of one skilled in the art to affect such feature, structure, or characteristic in connection with other implementations whether or not explicitly described herein.

The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the term “runtime” corresponds to any behavior that is dynamically determined during an execution of a software application or at least a portion of software application.

In some embodiments, the inventive specially programmed computing systems with associated devices are configured to operate in the distributed network environment, communicating over a suitable data communication network (e.g., the Internet, etc.) and utilizing at least one suitable data communication protocol (e.g., IPX/SPX, X.25, AX.25, AppleTalk™, TCP/IP (e.g., HTTP), etc.). Of note, the embodiments described herein may, of course, be implemented using any appropriate hardware and/or computing software languages. In this regard, those of ordinary skill in the art are well versed in the type of computer hardware that may be used, the type of computer programming techniques that may be used (e.g., object oriented programming), and the type of computer programming languages that may be used (e.g., C++, Objective-C, Swift, Java, Javascript). The aforementioned examples are, of course, illustrative and not restrictive.

The material disclosed herein may be implemented in software or firmware or a combination of them or as instructions stored on a machine-readable medium, which may be read and executed by one or more processors. As used herein, the machine-readable medium may include any medium and/or mechanism for storing or transmitting information in a form readable by a machine (e.g., a computing device). By way of example, and not limitation, the machine-readable medium may comprise computer readable storage media, for tangible or fixed storage of data, or communication media for transient interpretation of code-containing signals. Machine-readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Machine-readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, flash memory storage, or any other physical or material medium which can be used to tangibly store the desired information or data or instructions, including but not limited to electrical, optical, acoustical or other forms of propagated signals (e.g., carrier waves, infrared signals, digital signals, etc.), and which can be accessed by a computer or processor.

In another form, a non-transitory article, such as non-volatile and non-removable computer readable media, may be used with any of the examples mentioned above or other examples except that it does not include a transitory signal per se. It does include those elements other than a signal per se that may hold data temporarily in a “transitory” fashion such as RAM and so forth. Various embodiments of the present disclosure may utilize on one or more distributed and/or centralized databases (e.g., data center).

As used herein, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Servers may vary widely in configuration or capabilities, but generally a server may include one or more central processing units and memory. A server may also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

As used herein, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network. Various types of devices may, for example, be made available to provide an interoperable capability for differing architectures or protocols. As one illustrative example, a router may provide a link between otherwise separate and independent LANs.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Software may refer to 1) libraries; and/or 2) software that runs over the internet or whose execution occurs within any type of network. Examples of software may include, but are not limited to, software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that actually make the logic or processor.

In some embodiments, the present disclosure is directed to apparatuses, systems, and methods that accurately predicts the zeta potential entirely from structure. For example, various embodiments of the present disclosure may be utilized in drug discovery and drug development, drug formulation conditions or in other applications such as but not limited to defense, agri-food production, bioprocessing, nutraceuticals, and industrial catalysis (for example, replacing conventional catalyst with enzymes that can operate in non-physiological conditions requires shelf-stable compositions).

Specifically, the various embodiments of the present disclosure calculate the zeta potential by modeling the molecule-solvent interface and is applicable for calculating the zeta potential during all electrokinetic phenomena.

Predicting the zeta potential from structure is direct prediction of zeta potential from molecular structure; therefore, measurement of electrophoretic mobility is not needed. Various embodiment of the present disclosure provide an ability to simulate protein mutations and predict zeta potential computationally with sufficient accuracy to facilitate target optimization. The direct prediction from structure requires the validation of assumption that electrokinetic slip plane coincides with hydrodynamic radius. The hydrodynamic radius can also be computed from molecular structure, allowing calculation of zeta potential from calculated electrophoretic mobility. For globular proteins, the slip plane position can be estimated by subtracting the protein radius from its computed hydrodynamic radius. Although the slip plane position and hydrodynamic radius differ in their theoretical definitions with the slip plane position being the position of the zeta potential during electrokinetic phenomena (e.g. electrophoresis) and the hydrodynamic radius being a radius pertaining to the edge of solvation during diffusion, they both represent the point where water and ions no longer adhere to a molecule.

Various embodiment of the present disclosure further improve safety in avoiding the risks of working with hazardous materials. It also saves raw materials (reducing costs) and saves time spent on research and development providing a faster route for products to get to the market.

Exemplary applications for predicting the zeta potential are using the zeta potential experimentally to assess adsorption processes and how well a molecule remains suspended in a specific solution. The zeta potential of a molecule is dependent on solution properties, specifically pH, temperature, ionic strength, relative dielectric, and ionic radii of ions. All of these properties can be varied and are considered during the development of formulation to allow for a molecule to remain suspended or hold a specific interaction with another molecule that adsorbs on its surface. The various embodiments of the present disclosure can identify the solution conditions that will give a molecular structure a certain zeta potential value allowing for reduction of time and resources spent at the lab bench. In the least, it provides a computational tool for guiding scientists to the appropriate conditions for a desired formulation. Thus, the various embodiments of the present disclosure hold applications in structure-based molecular design of drugs, proteins, and other molecules that hold a charge-dependent function while remaining suspended in solution.

Another exemplary application is identifying modulators (inhibitors/promoters) of protein-protein interaction (could draft a claim along the following lines). An exemplary method for identifying a modulator of an interface between two proteins comprising: identifying two protein known to interact; inserting computer software including the various embodiments of the present disclosure; introducing an agent predicted to modulate interaction at the interface; and evaluating the interaction at the interface in the presence or absence of the agent, wherein a change in the interaction in the presence of the agent identifies the agent as a modulator.

Some other applications of the various embodiments of the present disclosure includes but not limited to using the predictive power of the software to improve pre-existing compositions of e.g. therapeutics (this is particularly significant when dealing with an unstable therapeutic) and using the predictive power of the software to generate a new composition having good stability—the new composition could be formulated for a novel compound/agent. In some situations with respect to compositions, wherein there are multiple agents and it can be difficult to design a formulation that well suits each of the multiple, the various embodiments of the present disclosure designs a composition that improves stability and activity (even synergistic activity), and could even impact mode of delivery (e.g., intravenous vs oral) depending on the circumstances.

In some embodiments, an exemplary methodology of the present disclosure predicts the zeta (or electrokinetic) potential of a molecule from its crystal structure using specified solution properties, such as temperature, pH, relative dielectric, and ion concentrations and their ionic radii. In some embodiments, the exemplary methodology of the present disclosure models a Gouy-Chapman electric double layer over different molecular conformations sampled from molecular dynamics and captures the electrostatic potentials at the edge of their hydrodynamic radii. In some embodiments, the average of the captured potentials defines the zeta potential of the molecule in solution. In some embodiments, the exemplary methodology of the present disclosure allows for modeling of specific ion effects through implementing a Gouy-Chapman-Stern electric double layer, which will allow a more general definition of the zeta potential.

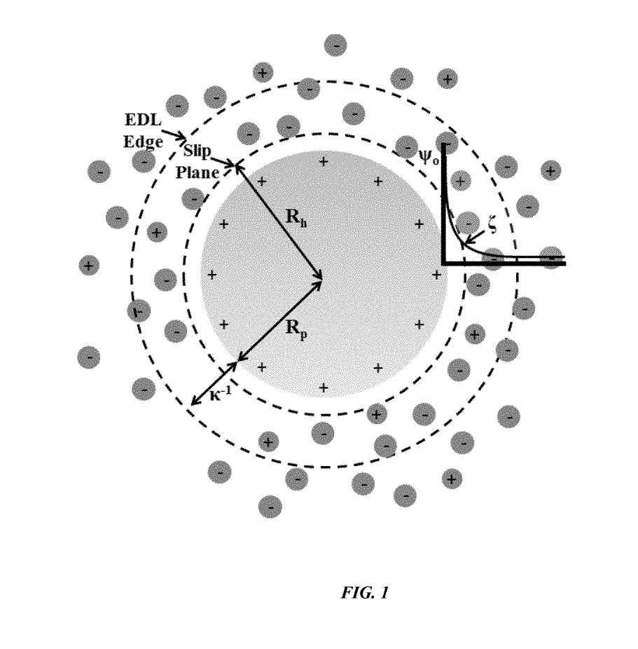

In some embodiments, the exemplary methodology of the present disclosure calculates the zeta potential of a protein from its molecular structure. The zeta potential is the effective charge density at the surface of a protein in solution. The zeta potential modeled using an electric double layer (EDL) (as shown in FIG. 1 and described in detail below): layers of charged solvent that forms to neutralize the charge of the protein's surface. The zeta potential is located at the slip plane: the boundary between the mobile solvent and immobile solvent attached to the surface. The location of the slip plan can be determined using the Gouy-Chapman-Stern EDL model. The hydrodynamic radius (Rh) is the radius of the protein plus the immobile solvation layer and can be calculated according to the Stokes-Einstein equation. The assumption of this disclosure is that the slip plane and the hydrodynamic radius coincide.

In some embodiments, the exemplary methodology of the present disclosure may include at least one or more the following steps:
1) Sample molecular conformations of the protein:
a) Prepare protein data bank (PDB) structure for molecular dynamics simulation,
b) Energetically minimize the PDB structure to optimize geometry,
c) Thermally excite the PDB structure (from OK) to induce thermal motion of solvent and protein, and
d) Simulate structure in solvent until it reaches a steady state and sample steady state conformations;
2) Estimate the slip plane location of each conformation: Estimate the Stokes-Einstein hydrodynamic radius (Rₕ), using the viscosity (η) of the solvent found in literature and the diffusivity (D) of the protein estimated using a hydrodynamic model:

$${R}_{h} = \frac{{k}_{b} T}{6 π η D} ;$$

3) Assign atomic charges and radii to each conformation: Protonate the protein using a pKa predictor at a specific pH and optimize the protonation state of titrateable residues (histidine, aspartic acid, glutamic acid, lysine and arginine) using software tools developed specifically for this purpose (for example, PROPKA 3.0 has been shown to be effective, although a number of equivalent tools are available including but not limited to MCCE, MEAD and UHBD);
4) Calculate electric potentials for each conformation: Solve the Poisson-Boltzmann equation over the structure to model the EDL (for example, by using the Applied Poisson Boltzmann Solver (APBS), but can be also accomplished using similar tools including but not limited to DelPhi, MEAD, ZAP, PBEQ, MIBPB, UHBD or ITPACT);
5) Calculate the zeta potential for each conformation: Generate a solvent-excluded surface (SES) for the protein and inflate to slip plane. Calculate the potential at each point on the slip plane surface and take the average to calculate the zeta potential for the conformation; and
6) Average the zeta potentials for all conformations.

The zeta potential affects the stability of molecules in dispersed systems such as foams (gas liquid), emulsions (liquid in liquid), and aerosols (solid or liquid in gas). Therefore, knowledge of the zeta potential enables prediction of the stability of certain drug formulations. Additionally, zeta potential would affect absorption onto surfaces, including pharmaceutical carriers for drug delivery.

One application of this tool would be to modify proteins to alter zeta potential without compromising the active site. In this application, a mutation would be made in the known protein structure, and the zeta potential would be calculated. In some embodiments, if the change in zeta potential is desirable, the protein could be synthesized. For example, a suitable zeta potential is qualitatively considered as one that is high enough for a stable dispersion—typically above 15 mV but lower than thermal energy (25 mV @ 25° C.). In some embodiments, protein aggregation is dependent on the zeta potential squared multiplied by the Debye length.

In some embodiments, the exemplary methodology of the present disclosure may include changing the solvent and increasing the concentration. In some embodiments, increased concentration is an important goal for increasing the amount of active drug per dose.

In some embodiments, the exemplary methodology of the present disclosure may experimentally validate of the assumption that the slip plane and hydrodynamic radius coincide using lysozyme. In some embodiments, diffusivity may be measured by dynamic light scattering and electrophoretic mobility measured using electrophoretic light scattering and phase analysis light scattering with a Zetasizer.

In some embodiments, the exemplary methodology of the present disclosure facilitates comparison of experimental and computational results on effects such as but not limited to effect of pH on electrophoretic mobility (test pKa prediction); effect of ion concentration on electrophoretic mobility (test slip plane prediction); effect of structural mutations on electrophoretic mobility; effect of temperature on electrophoretic mobility (capture transition in electrophoretic mobility).

For example, various embodiments detailed in the present disclosure may utilize one or more of the following approaches, but not limited to: in the first ‘Molecular Simulation’ step, using AMBER that can run calculations in parallel on a single workstation GPU. For example, various embodiments detailed in the present disclosure may run extended simulations in a practical amount of time (<1 day) on a single workstation (i.e., no computer cluster required). For example, various embodiments detailed in the present disclosure may utilize shell scripts that automate input/output management and connects all of the programs.

Various embodiments of the present disclosure determine the slip-plane position—i.e. the distance from a protein surface where the zeta potential is defined—without experimental measurements. Various embodiments of the present disclosure determine the slip plane based, at least in part, on the hydrodynamic radius, which in turn may be calculated from molecular structure using software tools such as, but not limited to HYDROPRO.

Slip Plane of a Protein Coincides with its Hydrodynamic Radius

The zeta potential (ζ) is the effective charge energy of a solvated protein, describing the magnitude of electrostatic interactions in solution. Predicting ζ from molecular structure would be useful to the structure-based molecular design of drugs, proteins and other molecules that hold charge dependent function while remaining suspended in solution. One challenge in predicting ζ is identifying the location of the slip plane (X_{SP}), a distance from the protein surface where ζ is theoretically defined. Various embodiment of the present disclosure estimate the X_{SP} by the Stokes-Einstein hydrodynamic radius (Rₕ), using globular hen egg white lysozyme as a model system. Although the X_{SP} and Rₕ differ in their theoretical definitions with the X_{SP} being the position of the ζ during electrokinetic phenomena (e.g. electrophoresis) and the Rₕ being a radius pertaining to the edge of solvation during diffusion, they both represent the point where water and ions no longer adhere to a molecule. Various embodiment of the present disclosure identify the range of ionic strength in which the X_{SP} can be modeled using the Stokes-Einstein equation defining a connection between diffusivity, hydration and ζ. In addition, various embodiment of the present disclosure may include determining the ζ from a protein crystal structure, which can be applied to optimize the dispersion stability of a protein solution.

In some embodiments, as disclosed herein, the zeta (ζ), or electrokinetic potential is the effective charge energy of a solvated solute. For example, it can be used to assess how well dispersed colloids remain in solution, and to model the electrokinetic behavior in adsorption processes. Protein-based therapeutics may be formulated at high concentrations that are prone to aggregation. Experimentally measured ζ has been applied to optimize therapeutic antibodies and other proteins for formulation conditions that promote long-term solution stability, and to study interactions between proteins with particles, materials and surfaces.

In some embodiments, the ability to predict ζ from the molecular structure of proteins would allow modeling of solubility as a parameter in computational protein design. In studies of protein self-assembly, unintuitive charge-dependent behavior may be observed due to a complicated balance between immediate and long-range electrostatic phenomena and between electrostatic and hydrodynamic processes. To model ζ, we treat the protein-solvent interface as an electric double layer (EDL), which is the collection of solvation layers that form around a protein in an attempt to neutralize its charge. Gouy and Chapman developed an EDL model where a molecule with a uniform surface charge is neutralized by a region of diffusing ions that encompass the molecular surface. The propagation of the surface potential and ion concentrations from the surface are defined by the Poisson-Boltzmann equation (PBE). In some embodiments, FIG. 1 depicts features of the EDL surrounding an idealized cationic spherical protein, and the electrostatic potential distribution extending into solution from the protein surface. For example, a hydration layer extends from the surface to the slip plane position (X_{SP}), similar to the Stern layer of the Gouy-Chapman-Stern (GCS) EDL. Plotted on the right, the surface potential (ψₒ) propagates outward into the cloud of ions treated as point charges immersed in a solvent with a constant relative dielectric. The zeta potential (ζ) is located at the slip plane, which is proposed to coincide with the hydrodynamic radius (Rₕ). Rₚ is the protein radius and κ⁻¹ is the Debye length.

For example, the depicted EDL assumes that the propagation of electrostatic potential and ion concentrations within the hydration layer are defined by the nonlinear PBE, unlike the GCS EDL, which applies a modified PBE to consider ion size constraints. ζ is weaker than the surface potential (ψₒ) and located at X_{SP}, which is somewhere in the cloud of diffusive ions less than a Debye length (κ⁻¹) away from the surface. In some embodiments, the X_{SP} represents the cutoff of an immobile layer of solvent (referred to as “hydration layer” in this work) adhering to the molecule. It is only a few molecular-sized layers thick. Ions adsorbed to the protein in this hydration layer can cause specific-ion effects that can be modeled with the GCS EDL.

In some embodiments, proteins provide an opportunity for EDL modeling where the molecular structures of their charged surfaces are known, and where changes in conformation can be studied experimentally or through simulation. For example, Hen egg white lysozyme, hereto referred to as lysozyme, is one such well-studied protein that can be used to evaluate EDL models.

In some embodiments, an obstacle to using atomic structure of proteins to estimate is the lack of general criteria for the location of the X_{SP}. Other studies have used the EDL edge defined by the Debye length, κ⁻¹, as X_{SP} for calculating ζ. However, at the κ⁻¹ motions of the ions are no longer determined by the surface potential, likely resulting in an underestimate of the calculated ζ. We hypothesize that a more accurate placement of X_{SP} is the radius of hydration (FIG. 1).

Theoretically, the Rₕ was derived as the radius of an uncharged sphere plus its immobile hydration layer undergoing diffusive motion. The X_{SP} and Rₕ differ in their theoretical definitions, with the X_{SP} being the position of the ζ during electrokinetic phenomena (e.g. electrophoresis) and the Rₕ being a radius pertaining to the edge of solvation during diffusion, defined by the Stokes-Einstein equation (Eq. 1).

$${R}_{h} = \frac{{k}_{b} T}{6 π η D} \qquad ( 1 )$$

where k_{b} is the Boltzmann constant, T is the absolute temperature, η is the pure solvent viscosity and D is the single particle diffusivity.

In addition to the choice of protein, the role of the counter-ion in determining EDL structure must be considered. Various embodiment of the present disclosure are directed to, without limitation, positively-charged protein, lysozyme, with weakly hydrated Cl counter-ions. In GCS theory on positively-charged particles, the center of anions makes up the inner Helmholtz plane, which is closer to the surface than the OHP, and can allow anions to sit on the molecular surface alongside water molecules. The concentration of counter-ions at the protein surface is physically limited by their size as they pack with water to coat the protein. In considering the finite size of ions, GCS theory is an improvement over GC theory that can model specific adsorption processes, which occur when an ion is attracted to a charged surface by more than just Coulombic forces. Multiple observations support that at pH 7, KCl is an indifferent electrolyte for lysozyme, dominated by Coulombic interactions. For example, key features of an electrostatic-dominated process such as an isoelectric point independent of ion concentration, and concentration dependent counter-ion binding are both observed in this system. Furthermore, small-angle X-ray scattering of lysozyme in KCl has also shown the Cl population in the nearest solvation layer increases with ion concentration. Therefore, chloride-lysozyme interactions can be modeled Coulombically, simplifying our analysis. As will be shown in this work, a modified GC EDL model applied to averaged lysozyme conformations provides an accurate representation of its electrokinetic behavior.

For example, various embodiments of the present disclosure the Stokes-Einstein equation to define the hydration layer within the modified GC EDL. Einstein originally derived Eq. 1 from the Navier-Stokes equation for dilute, non-charged spheres. For example, because lysozyme is charged at physiological pH, we must identify the effect that bearing a charge has on Rₕ. For example, at low ion concentrations, various embodiments of the present disclosure utilize diffusivity to show marked increases that lead to Rₕ being smaller than the physical size of the molecule itself—a hyper-diffusive regime. This increase in diffusivity is believed to result from long-range charge repulsion that accelerates diffusion as the κ⁻¹ increases. In some embodiments, various embodiments of the present disclosure establish the Stokes-Einstein regime (a range of ionic strengths) where Eq. 1 is valid for charge-bearing particle.

For example, various embodiments of the present disclosure utilize an effective hydrodynamic radius during electrophoresis (i.e. the electrophoretic radius) (Rₑ). Eq. 2a shows the relation between Rₑ, protein radius (Rₚ) and X_{SP}. Henry derived an equation for electrophoretic mobility (uₑ) accounting for electrophoretic retardation from the Poisson-Boltzmann and Navier-Stokes equations while assuming the ionic atmosphere surrounding the charged particle to remain in its equilibrium state (Eq. 2b). For example, various embodiments of the present disclosure may utilize Henry's equation that has been experimentally tested on nanometer to micron-scale polystyrene, gamboge and silica spheres. Eq. 2c expresses this relationship in terms of the protein net surface charge (Qe), knowing uₑ, the net valence of the protein (Q), and the pure solvent viscosity (η). Q is determined from controlling the solution pH and knowing the pKa values of the charged surface residues. η can be measured by a rheometer (see Table S1 of APPENDIX A); however, much data already exists on the viscosity of aqueous electrolyte solutions and thus we can use an empirical relationship (see Eq. S3 of APPENDIX A). For example, the Henry correction factor for electrophoretic retardation (f(κRₑ)) varies between 1 and 1.5, allowing us to calculate it as shown in Eq. 2d.

$$\begin{matrix}{R}_{e} = {R}_{p} + {X}_{SP} & ( 2 a ) \\ {u}_{e} = \frac{2 {ɛ}_{o} {ɛ}_{r} ζ f ( κ {R}_{e} )}{3 η ( \frac{f}{{f}_{o}} )} & ( 2 b ) \\ {R}_{e} = \frac{Qef ( κ {R}_{e} )}{6 π η {u}_{e} ( 1 + κ {R}_{e} ) ( \frac{f}{{f}_{o}} )} & ( 2 c ) \\ f ( κ {R}_{e} ) \approx 1 = \frac{1}{2 {( 1 + \frac{δ}{κ {R}_{e}} )}^{3}} , δ = \frac{2.5}{1 + 2 {e}^{- κ {R}_{e}}} & ( 2 d )\end{matrix}$$

where εₒ is vacuum permittivity, εᵣ is the solution relative dielectric constant, is the zeta potential, η is the pure solvent viscosity, κ is the inverse Debye length (see APPENDIX C) and

$$( \frac{f}{{f}_{o}} )$$

is a shape factor (1.17 for lysozyme).

For example, various embodiments of the present disclosure utilize a hypothesis that X_{SP}, Rₑ, Rₕ coincide, relating the position of the and the edge of solvation. For example, various embodiments of the present disclosure determine Rₑ and Rₕ to assess the similarity of the EDL during electrophoresis and diffusion. For example, various embodiments of the present disclosure utilize in the Stokes-Einstein regime, diffusivity alone to specify X_{SP}, and compute the ζ from the molecular structure of lysozyme. This assessment assumes the modified GC EDL model to be accurate for a protein in solution.

### Illustrative Examples of Utilizing Zeta Potential (ζ) Prediction (ZPRED).

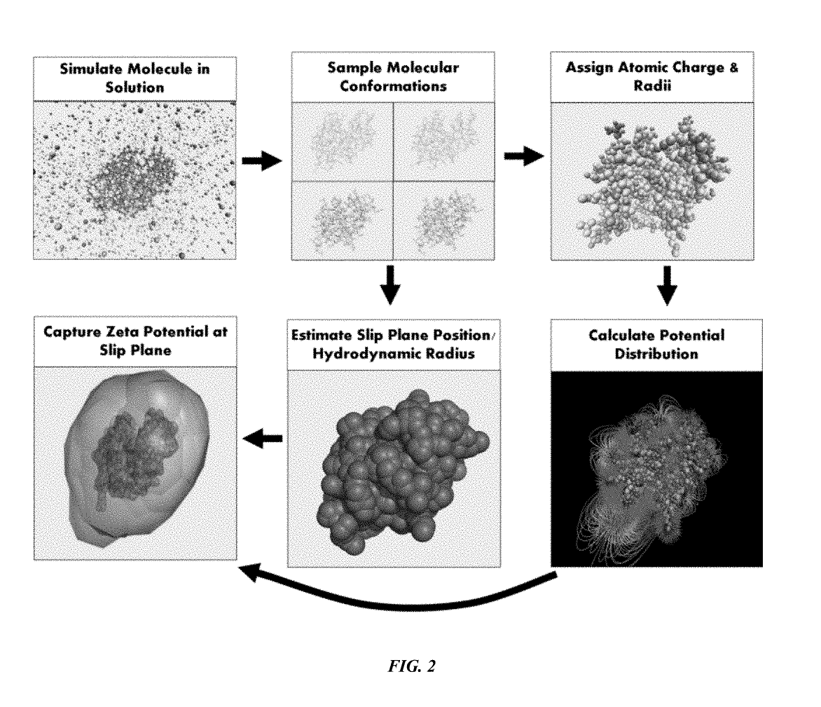

For example, various embodiments of the present disclosure utilize a modified Gouy-Chapman EDL model (FIG. 1) and computes the from a PDB structure through six primary steps (FIG. 2):
(1) sample molecular conformations of a PDB structure,
(2) estimate the slip plane position of each conformation,
(3) assign atomic charges and radii to each conformation,
(4) calculate electric potentials from each conformation propagating into implicit solvent,
(5) average electric potentials at the estimated slip plane to calculate the for each conformation, and
(6) calculate the of a PDB structure by averaging zeta potentials from the different conformations.
   As illustrated in FIG. 2, for example, various embodiments of the present disclosure utilize at least six steps to predict the ζ of a molecular structure. Each step provides a necessary piece of information accounting for the structural motions of the solvated molecule, its atomic charge distribution, its electric potential distribution into solution, and the distance from the molecule, where water and ions no longer adhere. For example, the slip plane position in the final step may be exaggerated for visual clarity.

1) Sample Molecular Conformations. At the 1st step, various embodiments of the present disclosure utilize the Amber 2015 molecular dynamics software suite to simulate the structural motions of the protein in solvent. In general, this may involve four steps, which are carried out computationally as described in APPENDIX E:
(1a) prepare the PDB structure for a molecular dynamics simulation,
(1b) energetically minimize the PDB structure,
(1c) thermally excite the PDB structure, and
(1d) simulate the structure in explicit solvent and sample conformations at structural steady-state.

In step 1a, crystal structures from the protein data bank are prepared using an Amber tool called, pdb4amber, which removes any water molecules present and protonates the crystal structure using another tool called, reduce. Prepared structures are loaded into a molecular dynamics simulation as an UNIT object, which is manipulated through the program teLeap. The teLeap program is run through a shell script called, tLeap, which takes an input file containing commands (see LEaP Input Command File for example). or example, in various embodiments of the present disclosure, input commands may specify force field parameters and generate initial topology and coordinates of the atoms of the prepared structure in a specified volume of solvent molecules. In general, globular (spherical) proteins are housed in water boxes extending 20 Å from the protein surface and fibrillar (cylindrical) proteins are housed in water boxes extending 30 Å away. For proteins, the ff14SB force field is used. Step 1b takes the generated topology and coordinate files and performs a molecular dynamics simulation using either sander or pmemd to energetically minimize the structure, which optimizes its geometry in solution (e.g. see Energy Minimization of Structure). In some embodiments, the coordinates of the optimized structure provide a starting point for the simulation of Step 1c. This step gradually heats the crystal structure from 0 K to a specified temperature, inducing thermal motion of the solvent and the protein (see Thermal Excitation). Step 1d is the main molecular dynamics simulation and uses the coordinates of the prepared heated structure as input (see Simulation in Solution). This simulation is run until the structure reaches a steady-state (e.g. about 100 nanoseconds) based on the root mean squared displacement of the protein backbone. The Amber tool, cpptraj, is necessary to use prior to calculating this displacement as it centers the entire trajectory of the solvent and protein coordinates around the protein's center of mass (see Post Simulation Processing). Once steady-state is reached, the protein switches between a limited number of molecular conformations, which are sampled based on the variation in the root mean squared displacement. The Amber coordinate files for these conformations are converted into PDB files using the ambpdb tool and the bres flag to ensure PDB-standard names are written to the file instead of Amber specific residue names (see Convert Coordinates to PDB Format).

2) Estimate the Slip Plane Position. In various embodiment of the present disclosure, the 2nd step, the position of the slip plane relative to the protein surface must be either determined from experimental data or estimated computationally. For example, various embodiments of the present disclosure utilize the Stokes-Einstein hydrodynamic radius (Rₕ) that may be determined from measured diffusivities, and the electrophoretic radius (Rₑ) determined from measured electrophoretic mobilities provide reasonable representations of the slip plane position. Thus, the slip plane position (X_{SP}) can be estimated by subtracting the protein radius (Rₚ) from a measured solvated radius, which should always be greater than or equal to the protein radius. It is important to note the Stokes-Einstein equation (Eq. 2) may be limited to relatively high salt concentrations, and thus, other methods for determining molecular size must be used, such as the electrophoretic radius (Rₑ) determined from electrophoretic mobility measurements (Eq. 2c).

For example, various embodiments of the present disclosure utilize estimating the X_{SP} computationally by estimating Rₚ and Rₕ, which physically represents the radius of a solvated molecule during diffusion. For globular proteins, Rₚ can be calculated as the average distance between the center of mass and the solvent-excluded surface (generated by MSMS (see APPENDIX F)) of the protein structure under assessment (see calcProteinRadius.cpp). As shown in Eq. 1, Rₕ depends on temperature, which is controlled by the user; leaving pure solvent viscosity and single particle diffusivity to be defined. A number of empirical relationships have been developed for the pure solvent viscosity of different salt solutions at varying temperatures in previous works (e.g., NaCl and KCl, NaH2PO4, Na2HPO4, Na3PO4, KH2PO4, K2HPO4, and K3PO4) and can be determined (see for example APPENDIX D). If values cannot be found, the viscosity of pure water (Eq. C2) can be used as an estimate since added salt only affects viscosity at higher ion concentrations. Single protein diffusivity can be computed with the software package, HYDROPRO (see APPENDIX G). HYDROPRO requires the protein structure, its specific volume (see getSpecificVolume.cpp), its molecular weight (see getMolecularWeight.cpp), temperature, pure solvent viscosity and pure solvent density as inputs. For example, various embodiments of the present disclosure utilize the software that is configured to use either a bead per atom or a bead per residue hydrodynamic model to determine the translational diffusivity of a single protein molecule. Each bead acts as a frictional center, and the frictional force it exerts on the solvent is calculated by Stoke's law. The frictional force between beads is also included in the overall calculation of the frictional force. Diffusivity is determined from the orientationally averaged frictional resistance in a simulated flow field. One technological shortcoming of HYDROPRO is that the software is best suited for smaller proteins (e.g. hen egg white lysozyme (6lyz), green fluorescent protein (2y0g), etc.) as it requires large, unobtainable amounts of memory for larger proteins, such as bovine serum albumin (3v03). For example, in various embodiments of the present disclosure, once viscosity and diffusivity values are obtained, Rₕ can be estimated by Eq. 1. For example, various embodiments of the present disclosure utilize estimated slip plane positions that are calculated by subtracting the protein radius from the estimated Rₕ. Once a slip plane position is determined, it is stored in a specifically designed data structure for later use in the 5th step of ZPRED.

3) Assign Atomic Charges and Radii. For example, various embodiments of the present disclosure, in the 3rd step, convert the PDB file containing the coordinates of the structure under assessment into a PQR file, which holds its coordinates in addition to atomic charge and radii values. For example, various embodiments of the present disclosure utilize the software package PDB2PQR that starts by checking the integrity of the structure (e.g., whether heavy atoms are missing or not) and then protonates it based on the pKa predictor, PROPKA, at a specified pH. Following protonation, for example, in various embodiments of the present disclosure, positions of hydrogens are determined by Monte Carlo optimization based on the global H-bonding network of the structure considering charge residue side chains and water-protein interactions. Once properly protonated, the structure is ready for electrostatic calculations in PQR format. A shell script for automating the usage of PROPKA and PDB2PQR can be found in APPENDIX I.

4) Calculate Electric Potential Distribution. In the 4th step, the protein's distribution of electrostatic potentials is computed by solving the Poisson-Boltzmann equation over the structure with the adaptive Poisson-Boltzmann solver (APBS) (see APPENDIX G). for example, various embodiments of the present disclosure utilize APBS that uses an adaptive finite element method which solves the Poisson-Boltzmann equation by iteratively adjusting the discretization of subsections of the problem domain. Subsections are allocated based on the error predicted from larger encompassing subsections initially starting with the entire problem domain. For example, various embodiments of the present disclosure utilize APBS that divides the problem into two regions of different dielectrics: the protein (e.g., dielectric of 2 to 4) and solvent (dielectric based on solvent and temperature). For example, various embodiments of the present disclosure utilize the two regions that are separated by a solvent-accessible surface generated over the protein structure using the largest ion in the solvent. APBS treats the surrounding solution as an implicit solvent and stores calculated potentials in the OpenDX data format, which is a 3D uniform-spaced matrix that is compatible with a number of built-in APBS tools. Among these tools, the program called multivalue is of importance and used in the next step. As described previously, the Poisson-Boltzmann equation models the diffuse region of the EDL, and in this step in various embodiment of the present disclosure, the connection between EDL theory and application is made. By solving either the complete non-linear Poisson-Boltzmann equation or the linear version using the Debye-Huckel approximation over the protein structure, a Gouy-Chapman EDL model encompassing the protein is generated. In some embodiment of the present disclosure, to model the previously discussed specific ion effects, a Gouy-Chapman-Stern EDL model may be used. Generating a Gouy-Chapman-Stern EDL would require modifying the protein surface to include a stagnant layer holding some dielectric and then solving the Poisson-Boltzmann equation from the stagnant layer into the diffuse region of the EDL holding a different dielectric. This is referred to as the Stern-layer-modified Poisson-Boltzmann equation and a discussion of its implementation is held off for future work. Another way to generate the GCS EDL is through solving the size-modified Poisson-Boltzmann equation, which accounts for ion size. Once the electric potentials are generated in the EDL model, it is now time to capture the potentials composing the zeta potential at the slip plane.

5) Average Electric Potentials at Slip Plane. The 5th step first involves generating a solvent-excluded surface (SES) on the PDB structure using MSMS. The SES generated is composed of Cartesian coordinates and their normal vectors directed away from the protein surface (see APPENDIX E). The SES is inflated to the slip plane by translating its initial coordinates along their respective normal vector by the estimated slip plane distance from the 2nd step (see inflateVert.cpp). Then the APBS tool, multivalue, uses the APBS calculated potentials and the inflated coordinates to capture the electric potentials at each point. This requires converting the inflated coordinates into a comma separated vector (CSV) file format, which is simply done by writing each coordinate on its own line and delimiting by commas in a text file (see vert2csv.cpp). A zeta potential value for each conformation is computed by averaging the captured potentials at the inflated SES (see calcZetaOutput.cpp).

6) Calculate the Zeta Potential of a Molecule. Various embodiment of the present disclosure include the 6th step that completes the zeta potential prediction by averaging the zeta potentials determined from each conformation. For example, in various embodiments of the present disclosure, the resulting zeta potential value represents what would be expected from the structure in solution assuming the modified Gouy-Chapman EDL is applicable, which should be the case for weakly charged proteins in simple 1:1 electrolyte solutions.

Illustrative Examples of Assessing the Feasibility of Lysozyme for EDL Analysis.

For example, various embodiments of the present disclosure utilize the lysozyme-KCl solution interface at pH 7 for assessing the relation between diffusivity, hydration and ζ with varying ionic strength. Typically, as a structure, lysozyme is highly spherical holding asphericity and shape parameter values indicative that the molecule can be represented by a sphere. The average asphericity from the 20 conformations produced by molecular dynamics was 0.0514±0.03 and the average value for the shape parameter was 0.0196±0.02 (0 is a perfect sphere for both values). For example, various embodiments of the present disclosure utilize the analysis of the hydration of lysozyme by subtracting the Rₚ from the Rₕ. Also, the net valence of the protein remains at +8 and is independent of ion concentration indicating the surface charge distribution provides a comparable EDL foundation for the different ionic strengths.

Although lysozyme can form dimers at pH 7, for example, in various embodiments of the present disclosure, monomers were present following filtration as described in the supplement. DLS measurements alone were not sensitive to dimerization (see Fig. S2 of APPENDIX A). For example, various embodiments of the present disclosure determine the oligomerization state using PALS electrophoretic mobility measurements (see Fig. S1 of APPENDIX A). For example, in various embodiments of the present disclosure, all measurements may be performed immediately upon the addition of salt solutions and routinely checked by DLS to ensure monodispersity.

The hydration of lysozyme studied by NMR and X-ray diffraction indicates solvent mostly forms a monolayer over the surface with ordered water structures extending no more than ˜4.5 Å. This hydration layer thickness is consistent with our measurements of Rₕ determined from experimental diffusivities and Rₑ determined from electrophoretic mobilities. For example, various embodiments of the present disclosure utilize the Stokes-Einstein equation (Eq. 1) to connect diffusivity and hydration. As the hydration layer thickness is the distance from the surface to where solvent no longer adheres to the protein, it may be similar to the X_{SP}, where the exists. To assess how well the Stokes-Einstein relation provides an estimate of the hydration layer thickness, and thus the X_{SP}, it is first necessary to determine the range of ionic strength in which Eq. 1 is valid. For example, various embodiments of the present disclosure utilize a combined analysis of the diffusive and electrophoretic behavior.

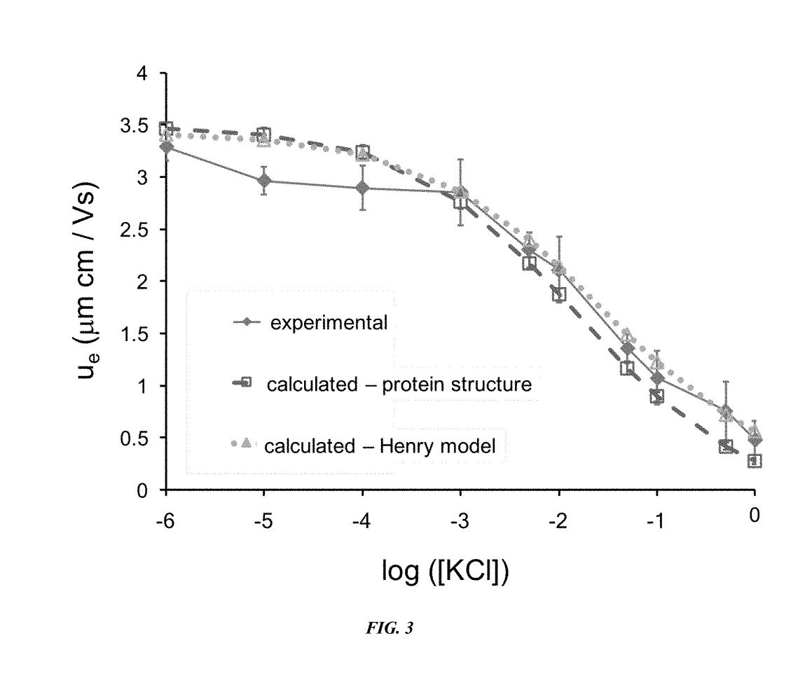

Electrophoretic Behavior of Lysozyme in KCl. Two approaches for calculating electrophoretic mobility—using the Henry model Eq. 2c, or using explicit protein structure—were compared to experimental values measured for multiple protein concentrations. Lysozyme concentrations were sufficiently low to allow negligible protein-protein interactions. Experimental uₑ consistently decreased with increasing ion concentration, which is expected of GC EDL behavior. Theoretical mobility values were calculated by rearrangement of the Henry model (Eq. 2c) using pure solvent viscosity values of KCl (Eq. S3) and a constant Rₑ value of lysozyme plus a monolayer of water (1.62+0.284 nm=1.904 nm). For example, various embodiments of the present disclosure utilize structure-derived mobility values using the exemplary ζ model can be calculated from the average of MD simulations of the lysozyme structure as detailed herein, and converted into electrophoretic mobilities (Eq. 2b) setting the shape factor equal to one. For example, various embodiments of the present disclosure utilize the Henry equation as an electrokinetic model under some conditions (i.e., only electrophoretic retardation is considered, no EDL polarization, and no surface conductivity. For example, in various embodiments, the ζ model used a constant hydrodynamic radius calculated from HYDROPRO (2.02 nm) to estimate the X_{SP}. A comparison of the two calculated and the experimental uₑ are shown in FIG. 3.

For example, various embodiments of the present disclosure utilize the electrophoretic mobilities of lysozyme based on the Henry model indicating the lysozyme-KCl EDL behaves like a GC EDL (FIG. 3). For example, various embodiments of the present disclosure utilize the Henry model that treats the protein as a sphere with a uniform surface charge and provides a standard for theoretical comparison with our detailed structure-based ζ model. For example, considering proteins are not perfectly spherical and hold a hydration layer that dampens their surface charge, measured protein mobilities are expected to be slightly lower than those predicted by the Henry model. For example, various embodiments of the present disclosure utilize at least one of models that represent the electrokinetic behavior of lysozyme in KCl, which indicates the modified GC EDL (structure model) is consistent with GC EDL theory (Henry model). This is significant as we have presented a theoretical validation of the GC EDL on an experimental crystal structure. It is important to note, dimerization can occur rapidly at the higher ionic strengths, and thus mobility values at the higher ion concentrations most likely represents a mixture of monomers and dimers. For example, various embodiments of the present disclosure may desire to minimize these effects (Fig. S1 of APPENDIX A).

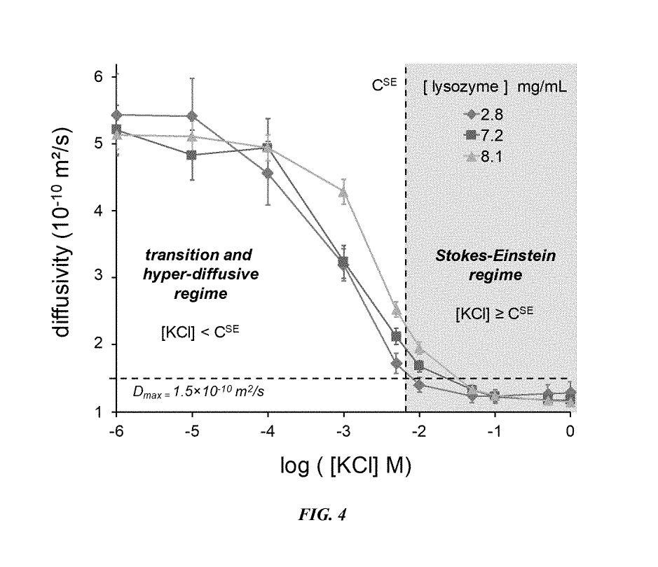

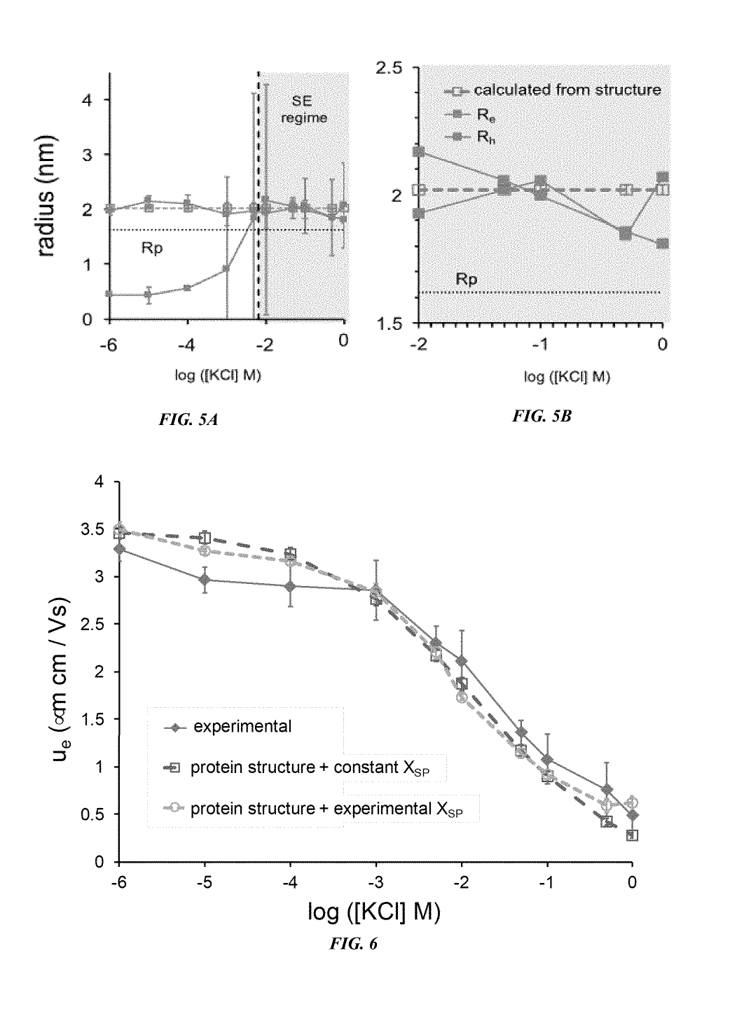

Diffusive Behavior of Lysozyme in KCl. Diffusivities of three concentrations of lysozyme were measured by DLS at a series of ionic strengths from micromolar to 1.0 M KCl (FIG. 4). For example, various embodiments of the present disclosure utilize the diffusion behavior that transitions between two different regimes with increasing ion concentration. The minimum KCl concentration defining the onset of the Stokes-Einstein regime where Eq. 1 is valid, denoted C^{SE}, was determined by comparison of Rₕ from diffusivity and Rₑ from electrophoretic mobility measurements (FIG. 5). Based on this analysis, the C^{SE} for KCl was interpolated to occur at 6.6 mM.

For example, in various embodiments of the present disclosure, at low ion concentrations, the hyper-diffusive regime exists in which diffusivity may be enhanced by inter-particle electrostatic phenomena. For example, in various embodiments of the present disclosure, the enhancement could be a change in structure. For example, various embodiments of the present disclosure are based in part on a mechanism by which, as counter-ions become incorporated in the EDL, they neutralize the electrostatic enhancement causing a transition. Once enough ions have become incorporated in the EDL to allow each lysozyme molecule to appear neutral to its neighbors (i.e. electroneutrality at the EDL edge), the Stokes-Einstein regime begins. In the Stokes-Einstein regime (i.e. [KCl]>C^{SE}), diffusivity and effective size can be related by the Stokes-Einstein equation (Eq. 1). In addition to the effects of ionic strength on the electrophoretic and diffusive behaviors through long-range electrostatic effects, in various embodiments of the present disclosure, may result in changes at the local scale in the nearest solvation layer around lysozyme in the Stokes Einstein regime.

EDL Contraction Affects Solvation in the Stokes-Einstein Regime. EDL contraction refers to the disintegration of the outer solvation layers with increasing ionic strength. This effect can be theoretically quantified with the Debye length, representing the EDL edge from the protein surface. As shown in FIG. 5A, analysis of protein diffusivity is only physically meaningful in the Stokes-Einstein regime. To estimate where the Stokes-Einstein regime becomes valid, we identified the ion concentration, where Rₕ and Rₑ first coincide. Experimental Rₕ values were calculated with Eq. 1 using experimentally determined single particle diffusivities (see Fig. S3) and the pure solvent viscosity (Eq. S3). For example, various embodiments of the present disclosure utilize experimental Rₑ values that are calculated with Eq. 2c using experimentally determined electrophoretic mobilities and the pure solvent viscosity. For comparison, various embodiments of the present disclosure utilize the HYDROPRO software to model single particle diffusivities based on the ensemble of lysozyme structures sampled by molecular dynamics. The structure of lysozyme during molecular dynamics remains compact with an average radius of 16.27±0.16 Å, calculated from the average center of mass to solvent excluded surface. This value represents the Rₚ, and is in agreement with past experimental findings. As the EDL contracts with increasing ion concentration in the Stokes-Einstein regime, experimental Rₕ values decrease (FIG. 5B). Experimental Rₕ decreases from 2.17 nm to 1.81 nm. For example, in various embodiments of the present disclosure, the computed hydrodynamic radii from HYDROPRO diffusivities remain constant at 2.02 nm, which is close to Rₕ for the Stokes-Einstein regime, and Rₑ values across the entire range of ionic strength.

In the Stokes-Einstein regime, calculated radii are all within error indicating agreement in the methods for determining molecular size and thus the hydration layer thickness. For example, in various embodiments of the present disclosure, this connection indicates the EDL of lysozyme is the same under both electrophoretic and diffusive conditions, supporting our hypothesis that the X_{SP} coincides with Rₕ. For example, various embodiments of the present disclosure utilize an estimation of the slip plane position from experimental that is subtracted the protein radius (Rₚ) from the measured Rₕ values in the Stokes-Einstein regime and Rₑ values outside of this regime (Eq. 3).

$${X}_{SP} = \{ \begin{matrix}{R}_{e} - {R}_{p} , {C}_{i} < {C}^{SE} \\ {R}_{h} - {R}_{p} , {C}_{i} \geq {C}^{SE}\end{matrix} \qquad ( 3 )$$

where X_{SP} is the slip plane position relative to the protein surface, Rₑ is the electrophoretic radius (Eq. 2c), Rₚ is protein radius, Cᵢ is the ion concentration, C^{SE} is the ion concentration at which the Stokes-Einstein regime begins, and Rₕ is the hydrodynamic radius (Eq. 1).

For example, in various embodiments of the present disclosure, Rₑ may be approximately equal to the radius of lysozyme plus a water molecule (1.62+0.284 nm), indicating a single layer of water of solvation. However, Rₕ may decrease with increasing ionic strength in the Stokes-Einstein regime, implying a shrinking hydration layer. For example, various embodiments of the present disclosure utilize applying th exemplary protein structure derived ζ model of the present disclosure using either a constant or a variable slip plane position.

ζ Analysis of the Slip Plane Estimates. For example, various embodiments of the present disclosure utilize an electrophoretic mobility, uₑ, determined from molecular structure with a calculated X_{SP} using HYDROPRO correlates quite well with experimentally measured uₑ (FIG. 3). For example, in various embodiments of the present disclosure, the calculated X_{SP} is constant, in contrast to observed changes in hydrodynamic radii based on diffusivity measurements. If we use X_{SP} values derived from experiment (Eq. 3) combined with a structure-based ζ model, we see little improvement in the correlation with directly observed uₑ values (FIG. 6). This suggests that for the given resolution of our instrument for determining uₑ, there is no advantage to including a variable X_{SP}. X_{SP} can be represented by the Rₕ experimentally and, for computational purposes, the X_{SP} can be approximated as constant over a wide range of ionic strengths.

For example, various embodiments of the present disclosure are based at least in part on the slip plane that a physical interface between bulk and constrained waters along the protein surface. For example, various embodiments of the present disclosure are configured to determine X_{SP} by utilizing diffusivity measurements in the Stokes-Einstein regime, thus connecting diffusivity, hydration and ζ. For example, various embodiments of the present disclosure may utilize experimental structures or atomic-resolution models In to predict ζ.

For example, various embodiments of the present disclosure may be directed to a number of protein targets, for a number of ion solution types, across a range of solution pH values, across a range of solution temperatures, and for the same protein with a series of point mutations. Various embodiment of the present disclosure include an optimization of solubility of a protein target.

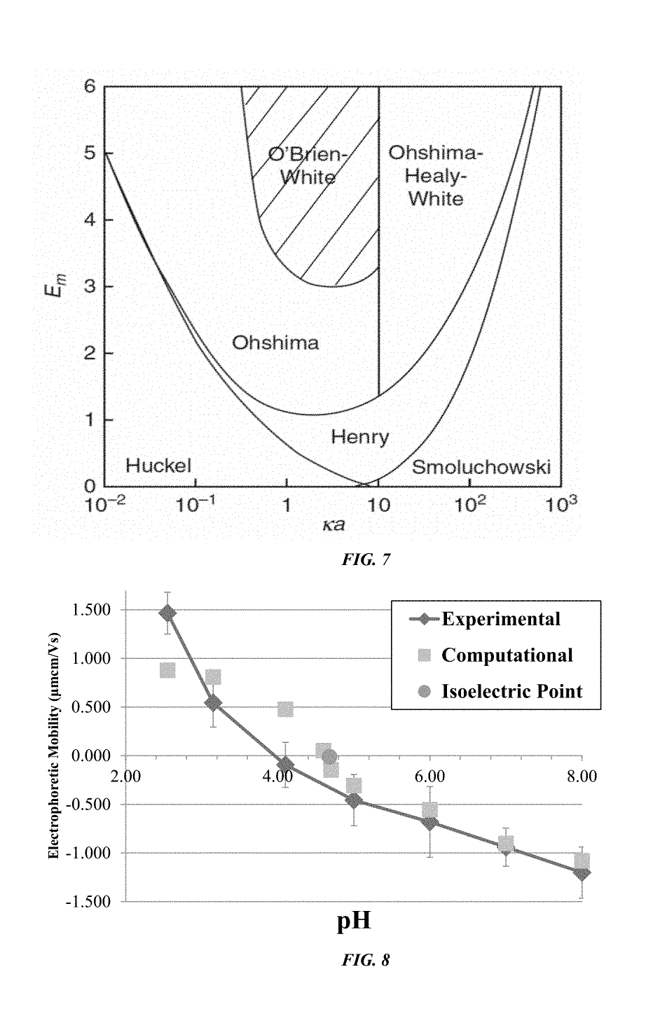

At least one technological problem is that the zeta potential is not directly measurable, but must be determined by an electrokinetic model relating it to at least one suitable measurable quantity, such as, without limitation, the electrophoretic mobility of electrophoresis. Typically, a method for getting at the zeta potential is electrophoresis; however, conversion of measured electrophoretic mobilities into a zeta potential value is complex and depends on the effective forces acting on the EDL when an electric field is perturbing it. For example, various embodiments of the present disclosure may be directed to electrokinetic models for converting electrophoretic mobility (uₑ) into a zeta potential (ζ), as shown in FIG. 7, and each account for different electrophoretic effects, which arise under different solution conditions. In FIG. 7, the dimensionless electrophoretic mobility (defined below) is plotted against the dimensionless electrokinetic radius (protein hydrodynamic radius divided by Debye length) to map the landscape, in which different effects arise.

$${E}_{m} = \frac{3 η e \vert {u}_{e} \vert}{2 {ɛ}_{r} {ɛ}_{o} {k}_{b} T} \qquad ( 4 )$$

where η is the pure solvent viscosity, μₑ is electrophoretic mobility, and the other terms hold their usual significance (see APPENDIX B for details)

For example, electrophoretic retardation is an effect to be modeled and is a viscous shear stress passed to the protein surface from oppositely moving counter-ions in the diffuse layer, which hinders the electrophoretic motion. This effect becomes more pronounced as ion concentration increases, which causes a larger electrokinetic radius due to a decreasing Debye length. As shown in FIG. 7, the Huckel equation (defined below with f_{ER}=1) accounts for the case of no electrophoretic retardation, and the Smoluchowski equation accounts for electrophoretic retardation at its maximum effect (defined below with f_{ER}=4). The Henry equation accounts for the transition between no and maximum electrophoretic retardation with the electrophoretic retardation correction factor (f_{ER}) formally defined in Eq. 5a.

$$\begin{matrix}{u}_{e} = \frac{2 {ɛ}_{r} {ɛ}_{o} ζ {f}_{ER}}{3 η} & ( 5 ) \\ {f}_{ER} = \frac{3}{2} ( 1 - {e}^{κ a} [ 5 {E}_{7} ( κ a ) - 2 {E}_{5} ( κ a ) ] ) & ( 5 a )\end{matrix}$$

where κ is inverse Debye length and Eₙ is the n-th order exponential integral (see APPENDIX A for definition and modeling approximation).

For example, another effect that is present predominately in particles with high charge is the relaxation effect. This effect refers to the distortion and effective polarization of the EDL that slightly neutralizes the electrokinetic charge reducing its attractive propulsion in the electric field, and thus hindering electrophoretic motion. The Ohshima approximation for Overbeek's expression for symmetrical electrolytes (see Eq. 6) accounts for the case of combined electrophoretic retardation and relaxation (see region of FIG. 7).

$$\begin{matrix}{u}_{e} = \frac{2 {ɛ}_{r} {ɛ}_{o} ζ f}{3 η} & ( 6 ) \\ f = {f}_{ER} - {( \frac{ze ζ}{{k}_{b} T} )}^{2} [ {f}_{3} + ( \frac{{m}_{+} + {m}_{-}}{2} ) {f}_{4} ] & ( 6 a ) \\ {f}_{e} = \frac{κ a ( κ a + 1.3 {e}^{( - 0.18 κ a )} + 2.5 )}{2 {( κ a + 1.2 {e}^{( - 7.4 κ a )} + 4.8 )}^{3}} & ( 6 b ) \\ {f}_{4} = \frac{9 κ a ( κ a + 5.2 {e}^{( - 3.9 κ a )} + 5.6 )}{8 {( κ a - 1.55 {e}^{( - 0.32 κ a )} + 6.02 )}^{3}} & ( 6 c ) \\ {m}_{\pm } = \frac{2 {ɛ}_{r} {ɛ}_{o} {k}_{b} T}{3 η {z}^{2} {e}^{2}} {λ}_{\pm } & ( 6 d )\end{matrix}$$

where λ_{±} is the ionic drag coefficient of cations and anions, which can be defined by either their limiting conductivities or their ionic radii.

Yet another effect that can arise with high ion concentrations is surface conductance in the diffuse layer. This effect refers to the excessive conductivity (relative to bulk solution) resulting from ion motion in the diffuse layer that distorts the applied electric field near the protein surface. For example, various embodiments of the present disclosure may be directed to the combined effects of electrophoretic retardation, relaxation and/or surface conductance that can be accurately modeled through solving the standard electrokinetic model, which is a system of coupled partial differential equations (specifically, the Navier-Stokes, Nernst-Planck, Poisson-Boltzmann, and/or Continuity equations). For example, Ohshima-Healy-White approximation solves the standard electrokinetic model by series expansion approximations and is only applicable at relatively high salt concentrations (see corresponding region of FIG. 7).

$$\begin{matrix}{u}_{e} = \frac{2 {ɛ}_{r} {ɛ}_{o} ζ f}{3 η} & ( 7 ) \\ f = 1 - \frac{2 AB}{\tilde{ζ} ( 1 + A )} + \frac{1}{\tilde{ζ} κ a} \{ W - X + Y - Z \} & ( 7 a ) \\ W = \frac{10 A}{1 + A} ( t + \frac{7 {t}^{2}}{20} + \frac{{t}^{3}}{9} ) - 12 C ( t + \frac{{t}^{3}}{9} ) & ( 7 b ) \\ X = 4 D ( 1 + \frac{2 {ɛ}_{r} {ɛ}_{o} {k}_{b} T}{η {ez}_{co}^{2} \vert {u}_{e , co} \vert} ) [ 1 - {e}^{- ( \frac{\tilde{ζ}}{2} )} ] & ( 7 c ) \\ Y = \frac{8 AB}{{( 1 + A )}^{2}} + \frac{6 \tilde{ζ}}{1 + A} ( \frac{2 {ɛ}_{r} {ɛ}_{o} {k}_{b} TD}{3 η {ez}_{co}^{2} \vert {u}_{e , co} \vert} + \frac{2 {ɛ}_{r} {ɛ}_{o} {k}_{b} TB}{3 η {ez}_{ctr}^{2} \vert {u}_{e , ctr} \vert} ) & ( 7 d ) \\ Z = \frac{24 A}{1 + A} ( \frac{2 {ɛ}_{r} {ɛ}_{o} {k}_{b} {TD}^{2}}{3 η {ez}_{co}^{2} \vert {u}_{e , co} \vert} + \frac{2 {ɛ}_{r} {ɛ}_{o} {k}_{b} {TB}^{2}}{3 η {ez}_{ctr}^{2} \vert {u}_{e , ctr} \vert ( 1 + A )} ) & ( 7 e ) \\ A = \frac{2}{κ a} ( 1 + \frac{2 {ɛ}_{r} {ɛ}_{o} {k}_{b} T}{η {ez}_{ctr}^{2} \vert {u}_{e , ctr} \vert} ) [ {e}^{( \frac{\tilde{ζ}}{2} )} - 1 ] & ( 7 f ) \\ B = ln ( 1 + {e}^{( \frac{\tilde{ζ}}{2} )} ) - ln ( 2 ) & ( 7 g ) \\ C = 1 - \frac{25}{3 ( κ a + 10 )} {e}^{- ( \frac{κ a \tilde{ζ}}{6 ( κ a + 6 )} )} & ( 7 h ) \\ D = ln ( 1 + {e}^{- ( \frac{\tilde{ζ}}{2} )} ) - ln ( 2 ) & ( 7 i ) \\ t = tanh ( \frac{\tilde{ζ}}{4} ) & ( 7 j ) \\ \tilde{ζ} = \frac{{z}_{ctr} e \vert ζ \vert}{{k}_{b} T} & ( 7 k )\end{matrix}$$

where z_{co} is the co-ion valence, u_{e,co} is the co-ion mobility, z_{ctr} is the counter-ion valence, and u_{e,tro} is the counter-ion mobility

For example, typically, the O'Brien-White algorithm numerically solves the standard electrokinetic model is cumbersome to use in contrast to the various embodiments of the present disclosure. For example, one or more models of the various embodiments of the present disclosure are configured to assume a stagnant layer of ions surround the main particle (in other words, stagnant layer conductance/mobile Stern layer is not considered). For example, various embodiments of the present disclosure may be configured to solve the above identified technological deficiency by accounting for all possibilities, a review of the possible effects and their modeling.

### Illustrative Examples of Electrophoretic Mobility Measurement.

For example, various embodiments of the present disclosure may be directed to measuring Electrophoretic mobilities by combined electrophoretic light scattering (ELS) and phase analysis light scattering (PALS) using a Malvern Zetasizer Nano ZS. For example, such measurement may employ a dip cell using the minimum measurement runs to minimize aggregation at the electrodes and provide adequate signal to noise ratios during both slow and fast field reversal. Samples may be checked for monodispersity, ensuring no aggregation by dynamic light scattering before and after measurements.

### Illustrative Examples of Experimental Validation Based on pH

FIG. 8 shows a comparison of experimental and computed electrophoretic mobilities: modeling the effect of pH experimental values (blue diamonds) for dimeric bovine serum albumin (BSA) at a concentration of 5 mg/mL and 20° C. show the typical trend of decreasing mobility with increasing pH citrate phosphate buffer was used to tailor the pH and induced a specific ion effect as shown by the shifted isoelectric point (IEP). For example, various embodiments of the present disclosure may be directed to testing the pKa prediction component in various embodiment of the present disclosure (PROPKA), by measuring and modeling electrophoretic mobilities at varying pH values. For example, an effect of pH to change the net charge of a protein based on the pKa values of its charged functional groups and thus the change in net charge of the protein may directly affect the electrophoretic mobility both in sign and magnitude. This effect was assessed using dimeric bovine serum albumin (BSA) in varying concentrations of citrate phosphate solution to adjust the pH from 2.55 to 8.00. 5 mg of BSA (LYSF, Worthington Biochemical Corporation) was dissolved in 1 mL of varying citrate phosphate solutions. Electrophoretic mobilities were measured at 20° C. by combined electrophoretic light scattering (ELS) and phase analysis light scattering (PALS) using a Malvern Zetasizer Nano ZS. Mobility measurement employed a dip cell using the minimum measurement runs to minimize aggregation at the electrodes and provide adequate signal to noise ratios during both slow and fast field reversal. The actual IEP of BSA is 4.68 in water; whereas it was found to be about 4.00 in our buffer. Computed values were determined by ZPRED as previously described using experimentally determined hydrodynamic radii. At pH below the IEP, BSA experiences a specific ion effect with phosphate, citrate or possibly both and deviates from computed values being the computation does not account for these effects. At pH above the IEP, BSA is negative and no longer experiences a specific ion effect with the anions. In this pH range, agreement between computed and experimental values can be seen (FIG. 8).

Illustrative examples of Experimental Validation based on Ion Concentration.

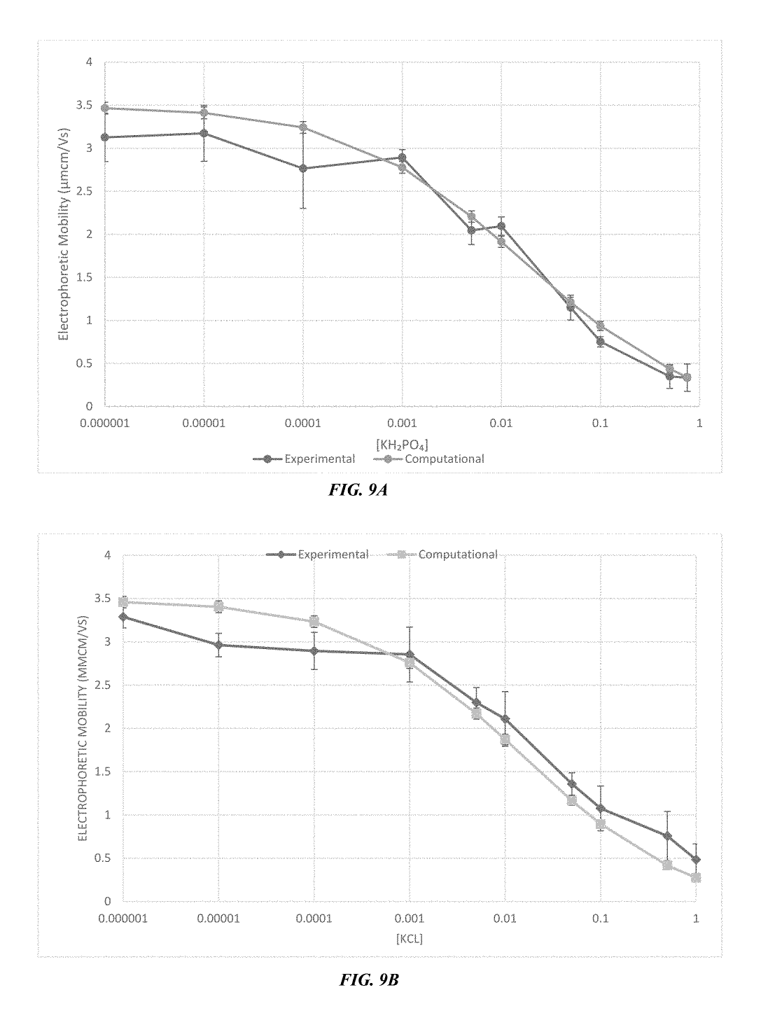

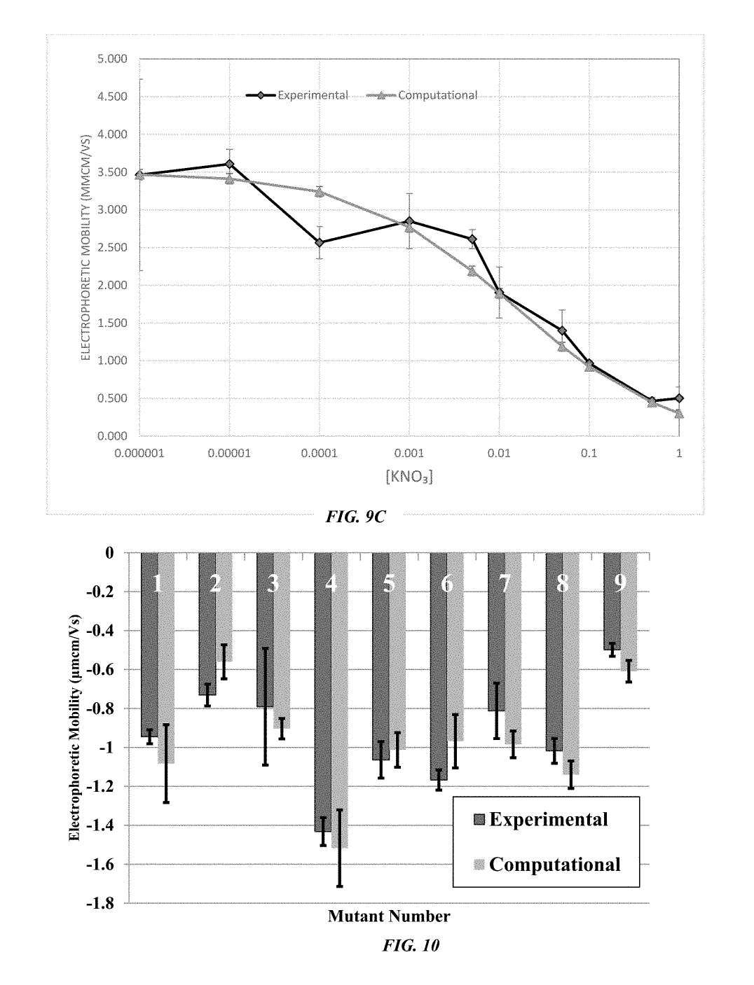

Regarding various embodiments of the present disclosure, FIGS. 9A-C compare experimental and computed electrophoretic mobilities from an exemplary ZPRED analysis of the present disclosure. Experimental values for monomeric lysozyme show the typical GC EDL trend of decreasing magnitudes with increasing ion concentration. Computed values were determined by ZPRED using HYDROPRO determined Rₕ values to estimate the X_{SP}. As shown, ZPRED is capable of adequately modeling the electrokinetic behavior of lysozyme across all ion concentrations for each of the three salts (KH₂PO₄, KCl, and KNO₃). To test the slip plane position prediction component (HYDROPRO), electrophoretic mobilities were measured and modeled at varying ion concentrations. For example, various embodiments of the present disclosure utilize the effect of ion concentration on electrophoretic mobility to reduce the mobility magnitude as ion concentration increases. This is the result of an increasing population of counter-ions that shield the electrokinetic charge of the protein, consequently reducing its acceleration in the applied electric field. This effect was assessed using monomeric hen egg white lysozyme in a wide range of concentrations of three different monovalent indifferent electrolytes (KH₂PO₄, KCl, and KNO₃). Lysozyme (LYSF, Worthington Biochemical Corporation) was dissolved in de-ionized water and filtered through 20 nm pore-size Anotop syringe filters to remove aggregates. Post-filtration lysozyme concentrations were determined with an Aviv Model 14DS spectrophotometer by UV absorption at 280 nm using α₂₈₀=2.64 mL/mg cm. In order to experimentally determine hydrodynamic radii, three protein concentrations (2.787, 7.246, and 8.064 mg/mL) were prepared in a wide range of indifferent electrolyte concentrations considering the balance between allowing the solution to remain concentrated enough for accurate light scattering measurements but dilute enough to ensure protein-protein interactions were negligible. Electrophoretic mobility measurements were performed as described in detail in the supplement. Computationally determined hydrodynamic radii were constant and thus yielded a constant slip plane position away from the molecular surface for each conformation of lysozyme (PDB id: 6LYZ) indicating the X_{SP} is a constant value for indifferent electrolytes.

### Illustrative Examples of Experimental Validation: Structural Mutations/Application to Molecular Design

FIG. 10 shows a comparison of experimental and computational electrophoretic mobilities: modeling the effect of structural mutations. Experimental mobilities were measured at 25° C. in a 50 mM Na₃Citrate buffer at a pH of 6.00. A key showing the residue mutations for each mutant number can be found in APPENDIX J. Computed values were determined from predicted zeta potentials using appropriate selection of the electrokinetic models shown in FIG. 7. Various embodiment of the present disclosure determine zeta potential from mutated structures generated from the wild type crystal structure (PDB id: 2y0g). As shown, prediction within experimental error can be achieved. Various embodiment of the present disclosure are applied in protein/drug design to accurately predict the zeta potential (and thus electrophoretic mobility) as, for example, without limitation, demonstrated on mutations of green fluorescent protein (GFP) (PDB id: 2Y0G) (APPENDIX K).

### Illustrative Examples of Experimental Validation: Rigid Rods and Flexible Chains.

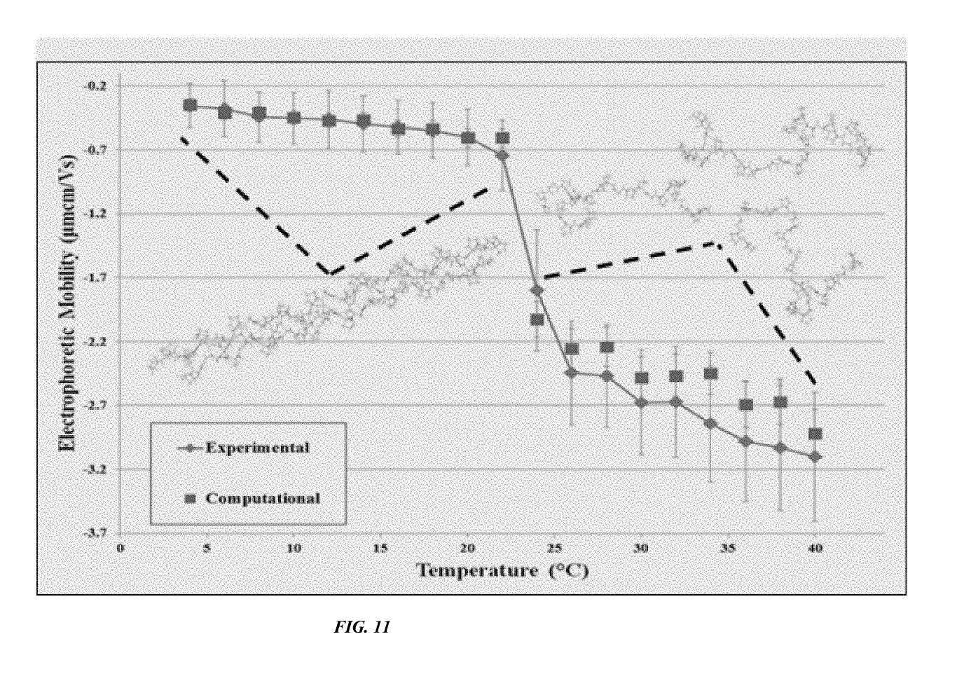

FIG. 11 compares experimental and computational electrophoretic mobilities of rigid rods and flexible chains. Experimental values for the melting collagen-like triple helix [(PPG)₁₀]₃ were measured in citrate phosphate buffer at pH 7.00. Measurements were taken over a wide temperature range around the triple helix's melting point (˜24° C.) to capture the transition in electrophoretic motion of the relatively rigid triple helices and the flexible PPG₁₀ chains. Computed values were determined from predicted zeta potentials using appropriate electrokinetic models shown in FIG. 7. Zeta potential prediction was applied to the [(PPG)₁₀]₃ crystal structure (PDB id: 1k6f) at temperatures below 24° C. and to an individual (PPG)₁₀ chain at higher temperatures. Various embodiment of the present disclosure are configured to calculate the zeta potential of cylindrical and flexible, chain-like proteins. The collagen-like triple helix, [(PPG)₁₀]₃, (PDB id: 1k6f) was used in this assessment.

### Illustrative Examples of Various Applications of the Zeta Potential Prediction

In some embodiments, the zeta potential may be used to assess the electrostatic stabilization of colloids, by assessing dispersion stability (i.e., how well separated a molecule remains in solution). The stability of weakly charged molecules in simple electrolytes is related to the zeta potential by the Eilers and Korff Rule, which states the loss of electrostatic stability occurs with a fast decline in the value of the product of the Debye length and zeta potential squared (κ⁻¹ζ²). In some embodiments, the value of κ⁻¹ζ² is proportional to interaction energy when dominated by electrostatic repulsion. Various embodiment of the present disclosure may utilize the Eilers and Korff Rule by running the exemplary zeta potential prediction tool for multiple ion concentrations and then identifying the solution conditions that induce a sharp decline in the value of κ⁻¹ζ4². In some embodiment, this identifies the critical coagulation concentration (the ion concentration inducing coagulation (aggregation) of a particular protein in solution), which can be compared to experimentally determined values.

For example, with the intention to control the stability of a protein in solution, various embodiments of the present disclosure provide a useful tool for designing either a solution environment for maintaining the electrostatic stabilization of a particular structure or a mutant structure that remains electrostatically stabilized in a particular solution environment. For example, various embodiments of the present disclosure utilize the incorporation of a Gouy-Chapman-Stern EDL model (size-modified Poisson-Boltzmann equation) on proteins with known specific ion effects, such as the isoelectric point shift shown in the BSA data (FIG. 8).

### Case Study—Optimization of Solution Stability of Biologics

Therapeutic peptides and proteins (biologics) are the most rapidly growing segment of therapeutic development, outpacing the market share growth of small-molecules. Composed of amino acids, typically, biologics are biocompatible and can achieve functional specificity and efficacy in many health-conditions not amenable to small-molecule drug design. Typically, various classes of biologics may include at least one of the following:
i) peptides—small proteins (usually less than 50 amino acids) that mimic hormones, cytokines or disrupt ligand-receptor interactions;
ii) enzyme replacement therapies (ERT)—many genetic diseases are caused by missing or malfunctioning enzymes (Gaucher disease, Fabry disease, Pompe disease), and ERTs involve administration of functional enzymes that are targeted to diseased tissues and rescue the missing activity; or
iii) therapeutic antibodies—a number of therapeutic antibodies are on the market (approximately 4-6 new antibodies are approved each year).

In all of these cases, there are fundamental technological issues related to the solution stability of biologics. Over time, proteins have the propensity to unfold and aggregate, limiting their shelf-life and potentially producing inactive or even toxic aggregate states.

As detailed herein, various embodiments of the present disclosure may be utilized in engineering variants of biologics with improved solution stability that would increase their half-life, lowering costs associated with storage and delivery. Furthermore, higher solution stability could promote increased biologic concentrations in the formulation—improving the dose/volume administered. This could result in fewer administrations to achieve the same therapeutic effect—which is particularly important for injections where administration is unpleasant, or in the case of injections into the cerebrospinal fluid, where smaller volumes of injections would lead to less local pain and neurological side-effects.

As detailed herein, various embodiments of the present disclosure may utilize the zeta-potential of protein(s) in, for example, without limitation, selecting appropriate amino acid substitution(s) that increase the zeta potential to improve solubility. For example, as mutations affect protein structure on the molecular scale, various embodiments of the present disclosure may allow to predict the effects of substitutions on zeta potential, allowing computational optimization of solubility.

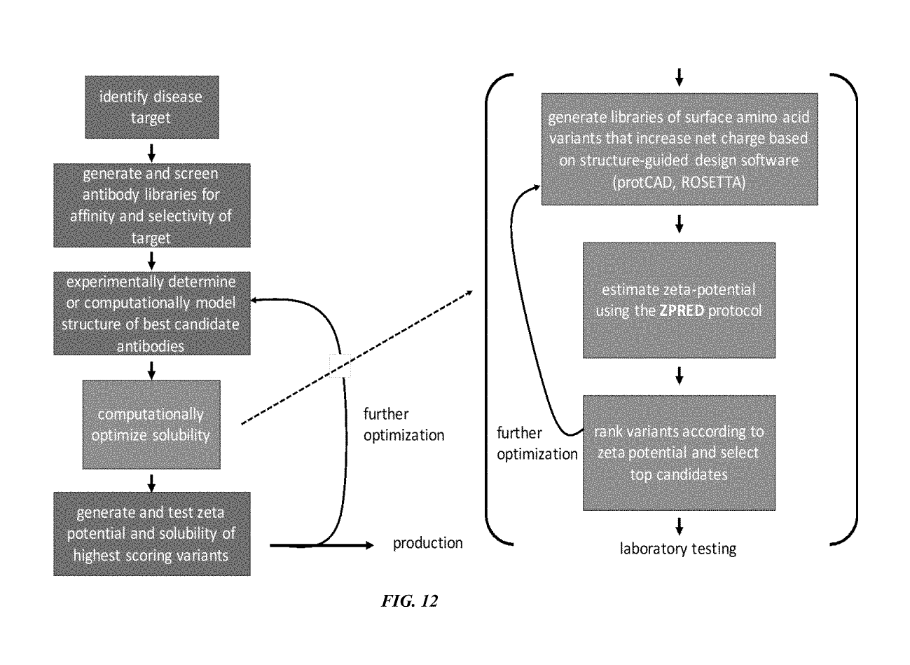

FIG. 12 show a case example of an illustrative application of the exemplary ZPRED implementation of the present disclosure that can be employed in biologics optimization.

In addition to varying amino acid sequence, the exemplary ZPRED implementation of the present disclosure can be used to estimate effects of solution conditions on one or more parameters (e.g., ionic strength, choice of salts, pH, etc.) in optimizing solution stability.

As detailed herein, various embodiments of the present disclosure may be utilized in applications directed to the optimization in the production and/or use of industrial enzymes. For example, optimizing solution stability may increase a shelf life of industrial enzymes, leading to lowering cost of storage, increasing the time which reagents could be used before they need to be replaced, and running reactions in smaller volumes, reducing costs of production.

In some embodiments, the present disclosure provides a method, comprising:

generating at least one modified compound having a modified structure;

wherein the at least one modified compound is related to an original compound having an original structure;

wherein the modified structure differs from the original structure;

wherein the at least one modified compound having the modified structure has an improved dissolution in a solution than the original compound having the original structure;

wherein the modified structure is determined based at least in part on:
i) sampling a plurality of molecular conformations of at least one of:
   1) the original compound having the original structure; and
   2) a plurality of candidate structures, wherein each candidate structure differs from the original compound in at least one conformational and structural change;
ii) for each molecular conformation of a respective sampled structure:
   1) estimating a hydrodynamic radius;
   2) estimating a slip plane position by subtracting a radius of the sampled structure from the estimated hydrodynamic radius;
   3) assigning atomic charges and radii to the respective molecular conformation of the respective sampled structure;
   4) determining potentials of the respective molecular conformation of the respective sampled structure in a simulated solution environment of the solution, at a solvent-excluded surface (SES) on the respective sampled structure inflated to the estimated slip plane which coincides with a calculated or measured hydrodynamic radius; and
   5) calculating a zeta potential of the respective molecular conformation of the respective sampled structure by averaging the determined potentials at the inflated SES;
iii) calculating an average zeta potential of each sampled structure by averaging the calculated zeta potentials of the plurality of molecular conformations of the respective sampled structure;
iv) comparing average zeta potentials of the plurality of candidate structures among each other and to an average zeta potential of the original compound; and
v) determining, based on the comparing at step (iv), at least one desired candidate structure;
vi) wherein the at least one desired candidate structure, based on a respective average zeta potential, is expected to have a higher solubility in the solution than at least one other candidate structure of the plurality of candidate structures and the original compound; and
vii) wherein the at least one desired candidate structure is the modified structure of the modified compound.

In some embodiments, the present disclosure provides the method further comprising design of the solution conditions.

In some embodiments, the present disclosure provides the method wherein the compound is a protein.

In some embodiments, the present disclosure provides the method wherein the compound is an antibody.

In some embodiments, the present disclosure provides the method wherein the compound is a catalyst.

In some embodiments, the present disclosure provides the method wherein the compound is an enzyme.

In some embodiments, the present disclosure provides the method wherein the sampling the plurality of the molecular conformations of the sampled structure comprises:

preparing the sampled structure for a molecular dynamics simulation;

optimizing a geometry of the sampled structure by energetically minimizing the sampled structure via a first molecular dynamics simulation;

thermally exciting the sampled structure;

performing a second molecular dynamics simulation with the sampled structure in the solution until the sampled structure reaches a steady-state; and

sampling a plurality of respective molecular conformations of the sampled structure.

In some embodiments, the present disclosure provides the method wherein the determining the at least one desired candidate structure comprises:

selecting the at least one desired candidate structure from the plurality of candidate structures.

In some embodiments, the present disclosure provides the method wherein the determining the at least one desired candidate structure comprises:

identifying at least one conformational and structural change to be made to at least one particular candidate structure of the plurality of candidate structures.

In some embodiments, the present disclosure provides the method wherein the slip plane position is determined based on calculation of the hydrodynamic radius.

In some embodiments, the present disclosure provides the method wherein the determining of the potentials of the respective molecular conformation of the respective sampled structure is at a solvent-excluded surface (SES) on the respective sampled structure inflated to the estimated slip plane.

Although the various embodiments of the present disclosure detailed herein describes or illustrates particular operations as occurring in a particular order, the present disclosure contemplates any suitable operations occurring in any suitable order. Moreover, the present disclosure contemplates any suitable operations being repeated one or more times in any suitable order. Although the present disclosure describes or illustrates particular operations as occurring in sequence, the present disclosure contemplates any suitable operations occurring at substantially the same time, where appropriate. Any suitable operation or sequence of operations described or illustrated herein may be interrupted, suspended, or otherwise controlled by another process, such as an operating system or kernel, where appropriate. The acts can operate in an operating system environment or as stand-alone routines occupying all or a substantial part of the system processing.

All examples and conditional language recited herein are intended for pedagogical purposes to aid the reader in understanding the principles of various embodiments of the present disclosure and the concepts contributed by the inventor to furthering the art, and are to be construed as being without limitation to such specifically recited examples and conditions. Moreover, all statements herein reciting principles, aspects, and various embodiments of the present disclosure, as well as specific examples thereof, are intended to encompass both structural and functional equivalents thereof. Additionally, it is intended that such equivalents include equivalents that perform the same.

What is claimed is:

1. A method, comprising:
obtaining at least one modified compound having a modified structure;
wherein the at least one modified compound is related to an original compound having an original structure;
wherein the modified structure differs from the original structure;
wherein the at least one modified compound having the modified structure has an improved dissolution in a solution than the original compound having the original structure;
wherein the modified structure is determined based at least in part on:
i) sampling a plurality of molecular conformations of at least one of:
1) the original compound having the original structure or
2) a plurality of candidate structures;
wherein each candidate structure differs from the original compound in at least one conformational change, at least one structural change, or both;
ii) for each molecular conformation of a respective sampled structure:
1) estimating, by a processor, a hydrodynamic radius;
2) estimating, by the processor, a slip plane position by subtracting a radius of the sampled structure from the estimated hydrodynamic radius;
3) assigning, by the processor, atomic charges and radii to the respective molecular conformation of the respective sampled structure;
4) determining, by the processor, potentials of the respective molecular conformation of the respective sampled structure in a simulated solution environment of the solution, at a solvent-excluded surface (SES) on the respective sampled structure inflated to the estimated slip plane which coincides with the estimated hydrodynamic radius or a measured hydrodynamic radius; and
5) calculating a zeta potential of the respective molecular conformation of the respective sampled structure by averaging the determined potentials at the inflated SES;
iii) calculating, by the processor, an average zeta potential of each sampled structure by averaging the calculated zeta potentials of the plurality of molecular conformations of the respective sampled structure;
iv) comparing, by the processor, average zeta potentials of the plurality of candidate structures among each other and to an average zeta potential of the original compound; and
v) determining, by the processor, based on the comparing at step (iv), at least one desired candidate structure;
vi) wherein the at least one desired candidate structure, based on a respective average zeta potential, is expected to have a higher solubility in the solution than at least one other candidate structure of the plurality of candidate structures and the original compound;
vii) wherein the at least one desired candidate structure is the modified structure of the modified compound; and
viii) adapting a desired compound having the at least one desired candidate structure to the solution.

2. The method of claim 1, wherein the method further comprises:
determining, by the processor, one or more solution conditions of the solution.

3. The method of claim 1, wherein the original compound is a protein.

4. The method of claim 1, wherein the original compound is an antibody.

5. The method of claim 1, wherein the original compound is a catalyst.

6. The method of claim 1, wherein the original compound is an enzyme.

7. The method of claim 1, wherein the sampling the plurality of the molecular conformations of the sampled structure further comprises:
preparing the sampled structure for a molecular dynamics simulation;
optimizing, by the processor, a geometry of the sampled structure by energetically minimizing the sampled structure via a first molecular dynamics simulation;
thermally exciting the sampled structure;
performing, by the processor, a second molecular dynamics simulation with the sampled structure in the solution until the sampled structure reaches a steady-state; and
sampling, by the processor, the plurality of respective molecular conformations of the sampled structure.

8. The method of claim 1, wherein the determining the at least one desired candidate structure further comprises:
selecting, by the processor, the at least one desired candidate structure from the plurality of candidate structures.

9. The method of claim 1, wherein the determining the at least one desired candidate structure further comprises:
identifying, by the processor, the at least one conformational change, the at least one structural change, or both, to be made to at least one particular candidate structure of the plurality of candidate structures.

10. The method of claim 1, wherein the slip plane position is determined based, at least in part, on the estimated hydrodynamic radius.

11. A system, comprising:
at least one specialized computer machine, comprising:
a non-transient memory, electronically storing particular computer executable program code; and
at least one computer processor which, when executing the particular program code, becomes a specifically programmed computer processor configured to at least:
determine a modified structure of at least one modified compound;
wherein the at least one modified compound is related to an original compound having an original structure;
wherein the modified structure differs from the original structure;
wherein the at least one modified compound having the modified structure has an improved dissolution in a solution than the original compound having the original structure;
wherein the determination of the modified structure of at least one modified compound comprises:
i) receiving sampling data of sampling a plurality of molecular conformations of at least one of:
1) the original compound having the original structure or
2) a plurality of candidate structures;
wherein each candidate structure differs from the original compound in at least one conformational change, at least one structural change, or both;
ii) for each molecular conformation of a respective sampled structure and based on the sampling data:
1) estimating a hydrodynamic radius;
2) estimating a slip plane position by subtracting a radius of the sampled structure from the estimated hydrodynamic radius;
3) assigning atomic charges and radii to the respective molecular conformation of the respective sampled structure;
4) determining potentials of the respective molecular conformation of the respective sampled structure in a simulated solution environment of the solution, at a solvent-excluded surface (SES) on the respective sampled structure inflated to the estimated slip plane which coincides with the estimated hydrodynamic radius or a measured hydrodynamic radius; and
5) calculating a zeta potential of the respective molecular conformation of the respective sampled structure by averaging the determined potentials at the inflated SES;
iii) calculating an average zeta potential of each sampled structure by averaging the calculated zeta potentials of the plurality of molecular conformations of the respective sampled structure;
iv) comparing average zeta potentials of the plurality of candidate structures among each other and to an average zeta potential of the original compound; and
v) determining based on the comparing at step (iv), at least one desired candidate structure;
vi) wherein the at least one desired candidate structure, based on a respective average zeta potential, is expected to have a higher solubility in the solution than at least one other candidate structure of the plurality of candidate structures and the original compound;
vii) wherein the at least one desired candidate structure is the modified structure of the modified compound; and
viii) at least one apparatus configured to adapt a desired compound having the at least one desired candidate structure to the solution.

12. The system of claim 11, wherein the specifically programmed computer processor is further configured to:
determine one or more solution conditions of the solution.

13. The system of claim 11, wherein the original compound is a protein.

14. The system of claim 11, wherein the original compound is an antibody.

15. The system of claim 11, wherein the original compound is a catalyst.

16. The system of claim 11, wherein the original compound is an enzyme.

17. The system of claim 11, wherein the sampling data of sampling the plurality of the molecular conformations of the sampled structure has been obtained by:
preparing the sampled structure for a molecular dynamics simulation;
optimizing a geometry of the sampled structure by energetically minimizing the sampled structure via a first molecular dynamics simulation;
thermally exciting the sampled structure;
performing a second molecular dynamics simulation with the sampled structure in the solution until the sampled structure reaches a steady-state; and
sampling the plurality of respective molecular conformations of the sampled structure.

18. The system of claim 11, wherein the specifically programmed computer processor is further configured to:
select the at least one desired candidate structure from the plurality of candidate structures.

19. The system of claim 11, wherein the specifically programmed computer processor is further configured to:
identify the at least one conformational change, the at least one structural change, or both, to be made to at least one particular candidate structure of the plurality of candidate structures.

20. The system of claim 11, wherein the slip plane position is determined based, at least in part, on the estimated hydrodynamic radius.
